(19) United States
(12) Patent Application Publication
(10) Pub. No.: US 2014/0010870 A1
(43) Pub. Date: Jan. 9, 2014

(54) PHARMACEUTICAL FORMULATION COMPRISING INOSITOL

(71) Applicant: Vittorio Unfer, Roma (IT)

(72) Inventor: Vittorio Unfer, Roma (IT)

(73) Assignee: LO.LI. PHARMA S.R.L., Roma (IT)

(21) Appl. No.: 14/006,582

(22) PCT Filed: Mar. 21, 2012
(86) PCT No.: PCT/EP12/54960

§ 371 Date: Sep. 20, 2013

(30) Foreign Application Priority Data
Mar. 22, 2011 (IT) MI2011A000445

(51) Int. Cl. A61K 31/047; A61K 45/06; A61K 9/50

(52) U.S. Cl. CPC A61K 31/047; A61K 9/5057; A61K 45/06
USPC 424/455; 514/729; 424/400

(57) ABSTRACT

The present invention relates to a pharmaceutical composition comprising solutions, suspensions of dispersions of inositol, or an isomer thereof, in a vehicle comprising gelatin, glycerol or mixtures thereof.

### FIELD OF THE INVENTION

The present invention relates to a pharmaceutical composition comprising inositol or an isomer thereof.

### PRIOR ART

Inositol, a chemical compound of formula C₆H₁₂O₆, is a hydrocarbon having a structure different to that of conventional sugars. It exists as nine possible isomers, of which the most important form, widely present in nature, is cis-1,2,3,5-trans-4,6-cyclohexanehexol, or myo-inositol.

Apart from myo-inositol, the other isomers that exist, even if only in minimal quantities, are scyllo-, muco-, D-chiro-, L-chiro-, neo-, allo-, epi- and cis-inositol.

Inositol has a molecular formula identical to that of glucose, although it differs in molecular structure. It is synthesized by the organism directly from glucose-6-phosphate, and for this reason it is frequently marked out as a pseudo-vitamin, forming part of the B group and called vitamin B8.

Inositol plays a fundamental role in the secondary messengers within the cells, in form of inositol phosphate, or as phosphatidyl-inositol (PI) or phosphatidyl-inositol phosphate (PIP).

Myo-inositol, in particular, participates in important processes, such as morphogenesis and cytogenesis, lipid synthesis, the constitution of the cell membrane, and cell growth (Berridge, M. J. “Inositol lipids and cell proliferation”, Biochim Biophys Acta 1987; 907: 33-45; Downes C. P. “The cellular function of myo-inositol”, Biochem Soc Trans 1989; 17:259-68). Scientific studies have demonstrated that myo-inositol is co-involved as a precursor in the synthesis of phospho-inosides and constitutes the system of transduction of signals of phosphatidyl-inositol (PtdIns), which is known to be co-involved in the regulation of various cellular functions, including gametogenesis, fertilization, cell proliferation and development, secretion, contraction and neural activity (Berridge, M. J., Irvine R. F. “Inositol phosphate and cell signalling”, Nature 1989; 306: 197-205; Divecha N., Irvine R. F. “Phospholipid signalling”, Cell 1995; 80:269-78; Herbert M., Gillespie, J. I., Murdoch, A. P. “Development of calcium signaling mechanisms during maturation of human oocytes” Mol Hum Reprod 1997; 3: 965-73; Berridge, M. J., Downes, C. P., Hanley, M. R. “Neural developmental actions of lithium: a unifying hypothesis”, Cell 1989; 59:411-9).

Since the work of Nestler et al. in 2000 (Nestler, J. E., Jakubowicz, D. J., Iuorno, M. J. “Role of inositol phosphoglycan mediators of insulin action in the polycystic ovary syndrome”, J Pediatr Endocrinol Metab. 2000; 13 Suppl 5:1295-8), more and more scientific evidence has accumulated that supports the physiological and therapeutic role of inositol, particularly in disorders linked to ovarian polycystosis.

Polycystic ovary syndrome or ovarian polycystosis, (PCOS), is a complex and heterogeneous disorder that affects 6-10% of women of reproductive age (Diamanti-Kandarakis E., Argyrakopoulou G., Economou F., Kandaraki E., Koutsilieris M. “Defects in insulin signaling pathways in ovarian steroidogenesis and other tissues in polycystic ovary syndrome (PCOS)”, J Steroid Biochem Mol Biol 2008; 109: 242-6), and is the principal cause of infertility (Dunaif A. “Insulin resistance and the polycystic ovary syndrome: mechanism and implications for pathogenesis”, Endocr Rev 1997; 18: 774-800). It is characterized principally by chronic anovulation, hyperandrogenism, an altered LH/FSH ratio (>2/3:1) and by characteristic structure of the polycystic ovary, verifiable by echographic analysis

It has now been widely demonstrated that insulin resistance is intrinsically linked to polycystic ovary syndrome. Indeed, insulin resistance is present in 50-70% of women with PCOS, independently of whether they are obese or of normal weight.

This disorder is considered the major factor in the pathogenesis of the syndrome ((Dunaif A. “Insulin resistance and the polycystic ovary syndrome: mechanism and implications for pathogenesis”, Endocr Rev 1997; 18: 774-800; Legro R. S., Gnatuk C. L., Kunselman A. R., Dunaif A. “Changes in glucose tolerance over time in women with polycystic ovary syndrome: a controlled study”, J Clin Endocrinol Metab 2005; 90: 3236-42). Women affected with PCOS are often obese, and this contributes to the development of insulin resistance. It is well known that insulin resistance frequently spontaneously develops in the direction of the onset of compensatory hyperinsulinemia, which leads to the hyperandrogenism typical of the syndrome (Poretsky L., Cataldo N., Rosenwaks Z., Guidice L. “The insulin-related ovarian regulatory system in health and disease” Endocr Rev 1999; 20: 532-82).

The excess of androgen hormones results in menstrual irregularity, the development of ovarian cysts, hirsutism, and other disorders related to those mentioned. In these women with PCOS, insulin resistance can furthermore increase the risk of developing glucose intolerance, type 2 diabetes mellitus, hypertension, dyslipidemia and cardiovascular problems (Legro R. S., Gnatuk C. L., Kunselman A. R., Dunaif A. “Changes in glucose tolerance over time in women with polycystic ovary syndrome: a controlled study”, J Clin Endocrinol Metab 2005; 90: 3236-42); Maitra A., Pingle R. R., Menon P. S., Naik V., Gokral J. S., Meherji P. K. “Dyslipidemia with particular regard to apolipoprotein profile in association with polycystic ovary syndrome: a study among Indian women” Int. J. Fertil Womens Med. 2001; 46: 271-7).

Hyperinsulinemia and hyperandrogenism are therefore the two principal characteristic factors of polycystic ovary syndrome, even if their cause-and-effect relationship is still the subject of debate (Dunaif A. “Changes in glucose tolerance over time in women with polycystic ovary syndrome: a controlled study”, J Clin Endocrinol Metab 2005; 90: 3236-42; Bremer A. A., Miller W. L. “The serine phosphorylation hypothesis of polycystic ovary syndrome: a unifying mechanism for hyperandrogenemia and insulin resistance” Fertil Steril 2008; 89: 1039-48).

However, much scientific evidence suggests that hyperinsulinemia is the primary factor contributing to ovarian hyperandrogenism. The pharmacologically achieved lowering of the insulin levels results in improvement of the hyperinsulinemia and hyperandrogenism, and restoration of normal ovarian function in women with PCOS (Dunaif A. “Changes in glucose tolerance over time in women with polycystic ovary syndrome: a controlled study”, J Clin Endocrinol Metab 2005; 90: 3236-42; Bremer A. A., Miller W. L. “The serine phosphorylation hypothesis of polycystic ovary syndrome: a unifying mechanism for hyperandrogenemia and insulin resistance” Fertil Steril 2008; 89: 1039-48).

PCOS women who are obese, and those of normal weight, present insulin resistance independently of the fat mass (Dunaif A., Segal K. R., Futterweit W., Dobrjansky A. “Profound peripheral insulin resistance, independent of obesity, in polycystic ovary syndrome” Diabetes 1989; 38: 1165-1174), and the scientific evidence suggests that a deficiency of this particular inositol phosphoglycan, containing D-chiro-inositol, may contribute to insulin resistance in individuals with glucose intolerance or type 2 diabetes (Kennington A. S., Hill C. R., Craig J., et al. “Low urinary chiro-inositol excretion in non-insulin-dependent diabetes mellitus” N. Engl. J. Med. 1990; 323: 373-378). Indeed, administration of substances having insulin-sensitizing activity, such as D-chiro-inositol (Iuorno M. J., Jakubowicz D. J., Baillargeon J. P., Dillon P., Gunn R. D., Allan G., Nestler J. E. “Effects of d-chiro-inositol in lean women the polycystic ovary syndrome” Endocr Pract 2002; 8: 417-423; Nestler J. E., Jakubowicz D. J., Reamer P., Gunn R.D., Allan G. “Ovulatory and metabolic effects of d-chiro-inositol in the polycystic ovary syndrome” N. Engl. J. Med. 1999; 340: 1314-1320) to PCOS women who are obese or those of normal weight increases the frequency of ovulation and reduces the levels of circulating androgens. In support of this hypothesis, some studies have demonstrated that oral administration of D-chiro-inositol improved glucose tolerance by reducing insulin levels in women with PCOS, whether of normal weight (Iuorno M. J., Jakubowicz D. J., Baillargeon J. P., Dillon P., Gunn R. D., Allan G., Nestler J. E. “Effects of d-chiro-inositol in lean women with the polycystic ovary syndrome” Endocr Pract 2002; 8: 417-423), or obese (Nestler J. E., Jakubowicz D. J., Reamer P., Gunn R. D., Allan G. “Ovulatory and metabolic effects of d-chiro-inositol in the polycystic ovary syndrome” N. Engl. J. Med. 1999; 340: 1314-1320). In such patients, it also reduced the levels of androgens, improved ovarian function, and led to a reduction in testosterone levels in the blood and to an improvement in metabolic parameters, such as arterial blood pressure and triglyceride levels in women with PCOS (Genazzani A. D., Lanzoni C., Ricchieri F., Jasonni V. M. “Myo-inositol administration positively affects hyperinsulinemia and hormonal parameters in overweight patients with polycystic ovary syndrome” Gynecol Endocrinol 2008; 24(3): 139-44).

Inositol is commonly used in clinical practice for treating the ovarian polycytosis syndrome. Various products containing inositol are commercially available, the pharmaceutical formulations of which are granulate or tablets.

During the research work performed on inositol, it was discovered that light, humidity, temperature, contact with oxygen, the pH, the production process, the presence of excipients, etc are degrading factors that can affect the inositol titer and the presence of yeasts and moulds.

### SUMMARY OF THE INVENTION

The aim of the present invention is to provide an easy-to-use formulation of inositol for oral use, wherein the inositol titer is stable for prolonged periods.

It is also the objective of the present invention to provide a formulation of inositol for oral use that enables a plasma inositol concentration to be achieved which is higher than that obtained with the forms currently available.

Another objective of the present invention is to provide a method of manufacturing a formulation of inositol for oral use that is both of stable inositol titer and easy to use.

One objective of the present invention is to provide a composition for the treatment of polycystic ovary syndrome.

### BRIEF DESCRIPTION OF THE DRAWING

FIG. 1 is a diagram showing the increase of the inositol concentration in plasma when inositol is administered in the form of powder or soft gel respectively.

### DETAILED DESCRIPTION OF THE INVENTION

These aims, and others which will be described below, have been achieved by means of a pharmaceutical composition comprising a solution, suspension or dispersion of inositol, or an isomer thereof, in a vehicle comprising gelatin, glycerol or mixtures thereof.

The aims of the present invention have also been achieved by means of said composition for use in the treatment and/or prevention of polycystic ovary syndrome, insulin resistance, hyperinsulinemia, hypoglycemia, hyperandrogenism, metabolic syndrome, dyslipidemia, type 2 diabetes mellitus and cardiovascular/cerebrovascular diseases; it is also used in Medically Assisted Procreation (MAP) therapies in order to improve oocyte quality, and to optimize ovarian hyperstimulation protocols; in particular to prevent ovarian hyperstimulation syndrome. Further beneficial effects have been observed on the classical symptoms of the menopause, such as: irritability, hypertension, osteoporosis, dyslipidemia, weight gain, hot flushes and aging of the skin.

The aims of the present invention have also been achieved by means of a process for the manufacture of said composition comprising the dissolution, suspension or dispersion of inositol, or of at least one of its isomers, and of at least one excipient and/or plastifier in a vehicle comprising glycerol, gelatin or mixtures thereof.

Within the scope of the present invention, semi-liquid phase means the various types of suspension with which it is possible to formulate inositol and any constituents present in the composition.

Within the scope of the present invention, “softgel” is used to mean a dosage form consisting of a gelatin-based shell that encloses a liquid filling, wherein the shell comprises a combination of gelatin, water, opacifier and plastifier such as glycerin and/or sorbitol. The liquid filling of the softgel comprises a uniform matrix composed of glycerol and inositol, and is enclosed by a shell as defined above, forming what is known as a “softgel pearl”.

In one aspect, the present invention relates to a pharmaceutical composition comprising a solution, suspension or dispersion of inositol, or an isomer thereof, in a vehicle comprising gelatin, glycerol or mixtures thereof.

Surprisingly, it was found that the pharmaceutical forms for oral administration, obtained according to the present invention, are clearly less sensitive to the various degrading influences described above for the known pharmaceutical forms, as can be noted from the data presented in the table.

TABLE
Stability data inositol titration.

| | Pharmaceutical form | Pharmaceutical form |
|---|---|---|
| Stability | powder | softgel |
| Time 0 | 120 | 120 |
| 6 months | 115 | 119 |
| 12 months | 111 | 119 |

In particular, the present invention comprises inositol-based pharmaceutical compositions in capsules, for example, in soft capsules of softgel, having uniform matrices which, as well as being free from micro-contaminations that can further auto-catalyse decomposition, determine additional advantages such as, for example, the elevated and more immediate bioavailability of the active principle within the gastrointestinal environment.

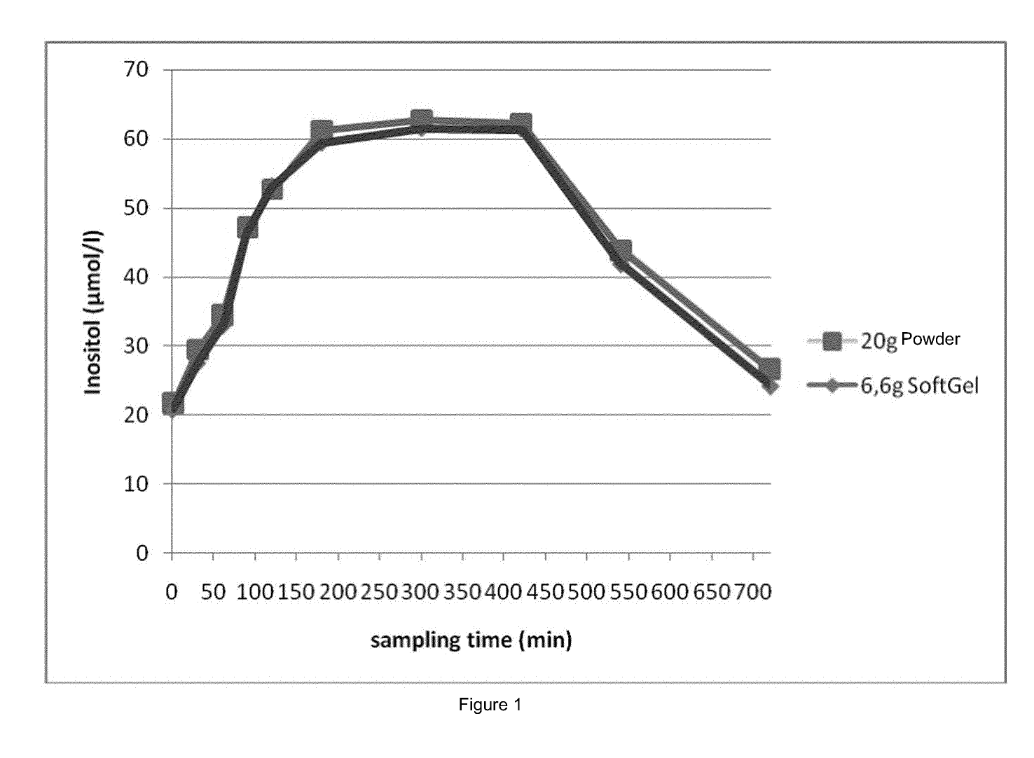

It was discovered in pilot studies that the pharmacokinetic parameters are surprisingly superior to those obtained with known formulations. In particular, one third of the doses of inositol, which was administered as soft gelatin capsules, induces an increase in the plasma concentration comparable to that induced by the powder, as can be seen in FIG. 1.

The swallowable, softgel pharmaceutical form of uniform matrix or in capsules, preferably in soft capsules (that is, that can be covered with a enteric coating decomposable, on the basis of the pH value, within the desired region of the gastrointestinal tract), may be composed of a shell that contains inositol and excipients, possibly in solid form or in a liquid or semi-liquid vehicle, together with supplementary excipients as necessary.

The composition according to the present invention is preferably a dosage form consisting of a gelatin-based shell and a filling of said shell, comprising said one solution, suspension or dispersion of inositol, or an isomer thereof, in a vehicle comprising gelatin, glycerol, ethanol or mixtures thereof.

In said composition, the dosage form is preferably a soft capsule or a softgel pearl.

In the composition according to the present invention, said shell is preferably coated with an outer coating that allows the release of inositol in the small intestine. Such a coating can be produced on the basis of the prior art in such a way as to break up substantially within the region of the small intestine, the principal location of inositol absorption.

In the composition according to the present invention, said shell is preferably coated with an outer coating that facilitates ingestion.

In a preferred embodiment, the composition according to the present invention is in the form of a softgel pearl of uniform matrix, comprising glycerol and inositol.

Furthermore, the hardness of the composition according to the present invention in the form of swallowable soft or softgel capsules of uniform matrix can be controlled on the basis of the capsule type or the uniform matrix of the swallowable softgel, which capsule type or uniform matrix are to be obtained by means of known plastifiers pharmacologically accepted for capsules, such as, for example, polyhydroxy alcohols, preferably glycerol, 1,2-propylene glycol, sorbitol solutions, etc.

In the case wherein the composition of the invention consists of softgel of uniform matrix, comprising both inositol and the possible excipients and/or plastifiers, such a structure allows a rapid release of the contents, from the envelope or from the matrix respectively, and therefore having a rapid release of the active principle, which has already been dissolved and/or dispersed.

The materials used to obtain the swallowable soft or softgel capsules of uniform matrix according to the present invention are common gelatins of so-called type A (elastic and soft capsule consisting of a shell of gelatin material containing a liquid or semi-liquid phase including, within it, the principal dissolved in gelatin, and/or glycerol), and type B (swallowable softgel pearl of uniform matrix composed of glycerol and of the principle) used within the pharmaceutical field, or methylcellulose, hydroxypropylmethylcellulose, calcium alginate, or other suitable materials of the pharmaceutical prior art that can also be used for the same purposes.

Said composition in the form of swallowable softgel pearls preferably also comprises type A or type B gelatin.

The composition of the invention preferably also comprises at least one active principle different from inositol, for example, folic acid, cocoa polyphenols, genistein, L-carnitine, L-arginine, vitamin E, selenium, N-acetylcysteine, and melatonin.

Further, optional common constituents of the soft or swallowable softgel capsules of uniform matrix, according to the present invention, are water and preservatives (such as antibacterials, antifungals, etc.), according to requirements.

The facultative excipients which may be used in preparing the uniform matrices of the swallowable softgels comprise the pharmacologically accepted constituents, such as, for example, solid additives as thickeners, which may become dissolved or dispersed in the liquid vehicle before or during gelification of the matrix, and/or preservatives.

The preferred vehicles are selected from glycerol, ethanol, polyethylene glycol or mixtures thereof, glycerol and mixtures of glycerol/ethanol. As non-limiting examples of gelatin, type A or B are preferable, while plastifiers may be added to modify the elasticity of the softgel in cases wherein the vehicles and/or the excipients previously referred to are not sufficient to achieve the desired result.

In particular, even in the case of the swallowable softgels of uniform matrix, the substances which provide multiple functions, for example glycerol (as a vehicle and/or plastifier) are especially preferred.

The composition according to the present invention preferably also comprises a plastifier.

More preferably, in said composition the plastifier is selected from glycerol, 1,2-polypropylene glycol, a sorbitol solution and mixtures thereof.

In the composition according to the invention, said vehicle preferably further comprises ethanol and glycerol.

Inositol is preferably present in the composition according to the invention in a quantity between 100 mg and 2 g. It was surprisingly found that the compositions of the present invention can contain relatively elevated quantities of inositol, or isomers thereof, without stability problems, thus enabling the administration of high doses to the patient with only one dose; administration in this pharmaceutical form furthermore facilitates the absorption of inositol.

In one preferred embodiment, in the composition according to the invention, the isomer of inositol is myo-inositol, scyllo-inositol, muco-inositol, D-chiro-inositol, neo-inositol, L-chiro-inositol, allo-inositol, epi-inositol, and cis-inositol or mixtures thereof.

In one aspect, the present invention relates to a process for manufacturing said pharmaceutical composition comprising the dissolution, suspension or dispersion of inositol, or of at least one isomer thereof, and of at least one excipient and/or plastifier, in a vehicle comprising glycerol, gelatin or mixtures thereof.

A further advantage of the composition of the invention in the form of swallowable softgel of uniform matrix derives from the fact that the pearl can be divided, at least in the case wherein they are not provided with enteric or similar coatings, on the recommendation of the doctor on behalf of the patient himself or herself, to allow further refinement of the daily dose beyond the units of the standard dose delivered by the pharmaceutical product. Moreover, in all the cases wherein solutions of active principles are used to obtain the gel matrix, the production of perfectly homogenous dosages is rendered especially easy.

In another aspect, the composition according to the invention is used in the treatment and/or prevention of pathologies of polycystic ovary syndrome, insulin resistance, hyperinsulinemia, hyperglycemia, hyperandrogenism, metabolic syndrome, dyslipidemia, type 2 diabetes mellitus and cardiovascular/cerebrovascular diseases; it is also used in Medically Assisted Procreation (MAP) therapies in order to improve oocyte quality, and to optimize ovarian hyperstimulation protocols; in particular to prevent the ovarian hyperstimulation syndrome.

Further beneficial effects have been observed on the classical symptoms of the menopause, such as: irritability, hypertension, osteoporosis, dyslipidemia, weight gain, hot flushes and aging of the skin.

1. Pharmaceutical composition comprising a solution, suspension or dispersion of inositol, or an isomer thereof, in a vehicle comprising gelatin, glycerol or mixtures thereof.

2. Composition according to claim 1, as a dosage form consisting of a gelatin-based shell and of a filling of said shell comprising said one solution, suspension or dispersion of inositol, or an isomer thereof, in a vehicle comprising gelatin, glycerol, ethanol or mixtures thereof.

3. Composition according to claim 2, wherein the dosage form is a soft capsule or a softgel pearl.

4. Composition according to claim 2, wherein said shell is coated with an outer coating that allows the release of inositol in the small intestine.

5. Composition according to claim 2, wherein said shell is coated with an outer coating which facilitates ingestion.

6. Composition according to claim 2, wherein said dosage form is a softgel pearl of uniform matrix comprising glycerol and inositol.

7. Composition according to claim 1, further comprising at least one active principle different from inositol.

8. Composition according to claim 7, wherein the isomer of inositol is myo-inositol, scyllo-inositol, muco-inositol, D-chiro-inositol, neo-inositol, L-chiro-inositol, allo-inositol, epi-inositol, and cis-inositol or mixtures thereof.

9. Composition according to claim 1, further comprising a plastifier in the shell.

10. Composition according to claim 9, wherein the plastifier is selected from glycerol, 1,2-propylene glycol, a sorbitol solution and mixtures thereof.

11. Composition according to claim 1, wherein said vehicle comprises ethanol and glycerol.

12. Composition according to claim 11, wherein said pharmaceutical composition is in a dosage form comprising inositol in a quantity between 100 mg and 2 g.

13. Process for manufacturing the composition according to claim 1, comprising the dissolution, suspension or dispersion of inositol, or of at least one isomer thereof, and of at least one excipient and/or plastifier in a vehicle comprising glycerol, gelatin or mixtures thereof.

14. Composition according to claim 1, for use in the treatment and/or prevention of polycystic ovary syndrome, insulin resistance, hyperinsulinemia, hyperglycemia, hyperandrogenism, metabolic syndrome, dyslipidemia, type 2 diabetes mellitus and cardiovascular/cerebrovascular diseases, in Medically Assisted Procreation (MAP) therapies in order to improve oocyte quality, and to optimize ovarian hyperstimulation protocols; in particular to prevent ovarian hyperstimulation syndrome, irritability, hypertension, osteoporosis, dyslipidemia, weight gain, hot flushes and aging of the skin.
